(19) United States
(12) Patent Application Publication
(10) Pub. No.: US 2025/0375152 A1
(43) Pub. Date: Dec. 11, 2025

(54) SYSTEM, METHOD, AND APPARATUS FOR DENTAL PATHOLOGY DETECTION ON X-RAY IMAGES IN VETERINARY ECOSYSTEMS

(71) Applicant: MARS, INCORPORATED, McLean, VA (US)

(72) Inventors: Fernando RODRIGUES JUNIOR, São Paulo (BR); Mark Justin PARKINSON, Newark, NJ (US); Joseph Conrad STACK, Chicago, IL (US)

(73) Assignee: MARS, INCORPORATED, McLean, VA (US)

(21) Appl. No.: 18/875,950

(22) PCT Filed: Jun. 16, 2023
(86) PCT No.: PCT/US2023/025581

§ 371 Date: Dec. 17, 2024

Related U.S. Application Data
(60) Provisional application No. 63/353,341, filed on Jun. 17, 2022.

(51) Int. Cl. A61B 5/00; G06T 7/00; G06T 7/12; G06T 7/143; G06T 7/194

(52) U.S. Cl. CPC A61B 5/4547; G06T 7/0012; G06T 7/12; G06T 7/143; G06T 7/194; A61B 2503/40; G06T 2207/10116; G06T 2207/20076; G06T 2207/20081; G06T 2207/30036

(57) ABSTRACT

In one embodiment, a method includes accessing a first image depicting an oral cavity of an animal, detecting multiple teeth of the animal from the first image based on machine-learning models, identifying each detected teeth based on a numbering protocol based on the machine-learning models, determining whether the tooth is healthy or has any dental pathology for each of the identified teeth based on the machine-learning models, localizing each tooth that has any pathology based on the numbering protocol, and generating a first report comprising a localization of each tooth that has any pathology.

### PRIORITY

This application claims the benefit, under 35 U.S.C. § 119(e), of U.S. Provisional Patent Application No. 63/353,341, filed 17 Jun. 2022, which is incorporated herein by reference.

### TECHNICAL FIELD

The embodiments described in the disclosure relate to dental pathology detection for pets. For example, some non-limiting embodiments relate to analyzing dental X-ray images to help detect a dental pathology of a pet.

### BACKGROUND

Veterinary dentistry is the field of dentistry applied to the care of animals. It is the art and science of prevention, diagnosis, and treatment of conditions, diseases, and disorders of the oral cavity, the maxillofacial region, and its associated structures as it relates to animals.

Machine learning (ML) is a field of inquiry devoted to understanding and building methods that “learn”, that is, methods that leverage data to improve performance on some set of tasks. It is seen as a part of artificial intelligence. Machine-learning algorithms build a model based on sample data, known as training data, in order to make predictions or decisions without being explicitly programmed to do so. Machine-learning algorithms are used in a wide variety of applications, such as in medicine, email filtering, speech recognition, and computer vision, where it is difficult or unfeasible to develop conventional algorithms to perform the needed task.

### BRIEF SUMMARY

The purpose and advantages of the disclosed subject matter will be set forth in and apparent from the description that follows, as well as will be learned by practice of the disclosed subject matter. Additional advantages of the disclosed subject matter will be realized and attained by the methods and systems particularly pointed out in the written description and claims hereof, as well as from the appended drawings.

To achieve these and other advantages, and in accordance with the purpose of the disclosed subject matter, as embodied and broadly described, the disclosed subject matter presents systems, methods, and apparatuses that can be used to collect, receive and/or analyze data. For example, certain non-limiting embodiments can be used to analyze dental pathology of pets.

In certain non-limiting embodiments, the disclosure describes a method for analyzing dental images (e.g., X-ray) of pets and determining dental pathology for pets accordingly. The method includes detecting teeth of a pet based on X-ray images of the oral cavity of the pet and numbering each of the detected teeth based on the Triadan system. In addition, the method includes determining whether each of the detected teeth is healthy or has any dental issues. The method further includes generating a report showing relevant information regarding the detected dental pathology of the pet.

In certain non-limiting embodiments, one or more computing systems can access a first image depicting an oral cavity associated with an animal. The computing systems can then detect, based on one or more machine-learning models, a plurality of teeth associated with the animal from the first image. The computing systems can then identify, based on the one or more machine-learning models, each of the detected teeth based on a numbering protocol. In certain non-limiting embodiments, the computing systems can further determine, for each of the identified teeth based on the one or more machine-learning models, whether the tooth is healthy or has any dental pathology. The computing systems can additionally localize each tooth that has any pathology based on the numbering protocol. In certain non-limiting embodiments, the computing systems can then generate a first report comprising a localization of each tooth that has any pathology.

In certain non-limiting embodiments, one or more computer-readable non-transitory storage media embodying software is operable when executed to access a first image depicting an oral cavity associated with an animal. The computer-readable non-transitory storage media embodying software is further operable when executed to detect, based on one or more machine-learning models, a plurality of teeth associated with the animal from the first image. The computer-readable non-transitory storage media embodying software is further operable when executed to identify, based on the one or more machine-learning models, each of the detected teeth based on a numbering protocol. In certain non-limiting embodiments, the computer-readable non-transitory storage media embodying software is further operable when executed to determine, for each of the identified teeth based on the one or more machine-learning models, whether the tooth is healthy or has any dental pathology. The computer-readable non-transitory storage media embodying software is further operable when executed to localize each tooth that has any pathology based on the numbering protocol. The computer-readable non-transitory storage media embodying software is further operable when executed to generate a first report comprising a localization of each tooth that has any pathology.

In certain non-limiting embodiments, a system can comprise one or more processors and a non-transitory memory coupled to the processors comprising instructions executable by the processors. The processors are operable when executing the instructions to access a first image depicting an oral cavity associated with an animal. The processors are further operable when executing the instructions to detect, based on one or more machine-learning models, a plurality of teeth associated with the animal from the first image. The processors are further operable when executing the instructions to identify, based on the one or more machine-learning models, each of the detected teeth based on a numbering protocol. The processors are further operable when executing the instructions to determine, for each of the identified teeth based on the one or more machine-learning models, whether the tooth is healthy or has any dental pathology. The processors are further operable when executing the instructions to localize each tooth that has any pathology based on the numbering protocol. The processors are further operable when executing the instructions to generate a first report comprising a localization of each tooth that has any pathology.

Furthermore, the disclosed embodiments of the methods, computer readable non-transitory storage media, and systems can have further non-limiting features as described below.

In certain non-limiting embodiments, the first image can comprise an X-ray image. The first image can be based on PNG format or DICOM format.

In certain non-limiting embodiments, the computing system can determine a quadrant for the first image based on the numbering protocol. The computing system can determine a view for the first image based on whether there is a composition of quadrants or not. In some embodiments, the view can comprise a lateral view or an occlusal view.

In certain non-limiting embodiments, detecting the plurality of teeth can comprise determining a plurality of box-coordinates for all possible teeth on the first image and calculating a probability score for each of the possible teeth based on the box-coordinates. In some embodiments, the probability score can indicate a likelihood of the corresponding possible tooth being a tooth.

In certain non-limiting embodiments, the computing systems can segment the plurality of detected teeth based on the one or more machine-learning model. In some embodiments, the segmentation can comprise generating a tooth boundary and a masked tooth without background for each of the plurality of detected teeth.

In certain non-limiting embodiments, the numbering protocol can be based on the Triadan system.

In certain non-limiting embodiments, identifying each of the detected teeth can be based on contextual information associated with each of the detected teeth.

In certain non-limiting embodiments, the one or more machine-learning models can comprise a first machine-learning model configured for identifying maxilla teeth and a second machine-learning model configured for identifying mandible teeth.

In certain non-limiting embodiments, the computing systems can determine, for each localized tooth, one or more pathologies associated with the tooth. The computing systems can then determine, for at least one of the one or more pathologies associated with each tooth, a level of grading.

In certain non-limiting embodiments, the computing systems can determine, based on the one or more machine-learning models, the first image comprises diagnostic information associated with dental pathology detection. In some embodiments, the diagnostic information can be based on one or more dental structures. In one feature, the one or more dental structures can be associated with a particular quadrant. In another feature, the one or more dental structures can be associated with a particular dental pathology.

In certain non-limiting embodiments, the computing systems can determine, based on the one or more machine-learning models, that the first image requires an alignment. The computing systems can further determine, based on the one or more machine-learning models, a degree to rotate the first image for the required alignment. The computing systems can further rotate, based on the one or more machine-learning models, the first image by the determined degree.

In certain non-limiting embodiments, the computing systems can receive, at the cloud computing system, a plurality of second images depicting the oral cavity associated with the animal. The computing systems can further process the plurality of second images in a parallel manner. In some embodiments, processing each of the plurality of second images can comprise using the one or more machine-learning models in a parallel manner to detect a plurality of teeth associated with the animal from each second image, identify each of the detected teeth based on the numbering protocol, determine, for each of the identified teeth, whether the tooth is healthy or has any dental pathology, and localize each tooth that has any pathology based on the numbering protocol. In one feature, processing the plurality of second images in the parallel manner can be based on logic generated based on one or more finite state machines. The computing systems can further generate a second report based on the first report and processing results of the plurality of second images.

It is to be understood that both the foregoing general description and the following detailed description are exemplary and are intended to provide further explanation of the disclosed subject matter claimed.

### BRIEF DESCRIPTION OF THE DRAWINGS

The foregoing and other objects, features, and advantages of the disclosure will be apparent from the following description of embodiments as illustrated in the accompanying drawings, in which reference characters refer to the same parts throughout the various views. The drawings are not necessarily to scale, emphasis instead being placed upon illustrating principles of the disclosure:

FIG. 1 illustrates example challenges faced with using artificial intelligence (AI) for pet dentistry.

FIG. 2 illustrates an example clustering pipeline for increasing the size of the tooth identification data in accordance with embodiments of the present disclosure.

FIGS. 3A-3B illustrate an example flow diagram for detecting pet dental pathology.

FIG. 4 illustrates an example comparison between a diagnostic image and a non-diagnostic image.

FIG. 5 illustrates another example comparison between a diagnostic image and a non-diagnostic image.

FIG. 6 illustrates an example diagnostic image for apical periodontitis prediction.

FIG. 7 illustrates example quadrants based on the Triadan system.

FIG. 8 illustrates an example tooth segmentation.

FIG. 9 illustrates an example teeth identification taking into account of context understanding.

FIG. 10 illustrates an example test experiment to evaluate teeth identification.

FIG. 11 illustrates an example context understanding for bone loss detection.

FIG. 12 illustrates example decoder attention maps.

FIG. 13A illustrates textual descriptions of the report.

FIG. 13B illustrates example evaluated images.

FIG. 14 illustrates an example flow diagram for parallel processing of multiple dental X-ray images in a cloud computing system.

FIG. 15 illustrates an example an example flow diagram for stateful orchestration.

FIG. 16 illustrates an example method for detecting tooth pathology.

FIG. 17 illustrates an example computer system.

### DESCRIPTION OF EXAMPLE EMBODIMENTS

The present disclosure will now be described more fully hereinafter with reference to the accompanying drawings, which form a part hereof, and which show, by way of illustration, certain example embodiments. Subject matter can, however, be embodied in a variety of different forms and, therefore, covered or claimed subject matter is intended to be construed as not being limited to any example embodiments set forth herein; example embodiments are provided merely to be illustrative. Likewise, a reasonably broad scope for claimed or covered subject matter is intended. Among other things, for example, subject matter can be embodied as methods, devices, components, and/or systems. Accordingly, embodiments can, for example, take the form of hardware, software, firmware or any combination thereof (other than software per se). The following detailed description is, therefore, not intended to be taken in a limiting sense.

The present disclosure provides systems, methods, and/or devices that can analyze pet dental pathology. The presently disclosed subject matter addresses needs associated with assessing the dental health of pets. The present disclosure presents a novel framework for localizing, identifying and grading teeth pathologies on canines and felines from X-ray images. The images are extracted from DICOM files and processed by a multi-stage algorithm. Specifically, a series of deep-learning based models use the global context to localize the teeth and identify them according to the Triadan system. The image is then sent to multiple models to detect dental pathologies. As an example and not by way of limitation, such dental pathologies include periodontal and endodontic diseases such as bone loss, apical periodontitis, inflammatory root resorption, crown fracture and more.

In the detailed description herein, references to “embodiment,” “an embodiment,” “one non-limiting embodiment,” “in various embodiments,” etc., indicate that the embodiment(s) described can include a particular feature, structure, or characteristic, but every embodiment might not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described. After reading the description, it will be apparent to one skilled in the relevant art(s) how to implement the disclosure in alternative embodiments.

In general, terminology can be understood at least in part from usage in context. For example, terms, such as “and”, “or”, or “and/or,” as used herein can include a variety of meanings that can depend at least in part upon the context in which such terms are used. Typically, “or” if used to associate a list, such as A, B or C, is intended to mean A, B, and C, here used in the inclusive sense, as well as A, B or C, here used in the exclusive sense. In addition, the term “one or more” as used herein, depending at least in part upon context, can be used to describe any feature, structure, or characteristic in a singular sense or can be used to describe combinations of features, structures or characteristics in a plural sense. Similarly, terms, such as “a,” “an,” or “the,” again, can be understood to convey a singular usage or to convey a plural usage, depending at least in part upon context. In addition, the term “based on” can be understood as not necessarily intended to convey an exclusive set of factors and can, instead, allow for existence of additional factors not necessarily expressly described, again, depending at least in part on context. As used herein, the words “may” and “can” are used in a permissive sense (i.e., meaning having the potential to), rather than the mandatory sense (i.e., meaning must). Similarly, the words “include”, “including”, and “includes” mean including but not limited to.

As used herein, the terms “comprises,” “comprising,” or any other variation thereof, are intended to cover a non-exclusive inclusion, such that a process, method, article, or apparatus that comprises a list of elements does not include only those elements but can include other elements not expressly listed or inherent to such process, method, article, or apparatus.

The terms “animal” or “pet” as used in accordance with the present disclosure can refer to domestic animals including, domestic dogs, domestic cats, horses, cows, ferrets, rabbits, pigs, rats, mice, gerbils, hamsters, goats, and the like. Domestic dogs and cats are particular non-limiting examples of pets. The term “animal” or “pet” as used in accordance with the present disclosure can also refer to wild animals, including, but not limited to bison, elk, deer, venison, duck, fowl, fish, and the like.

The term “pet owner” can include any person, organization, and/or collection of persons that owns and/or is responsible for any aspect of the care of a pet. For example, a “pet owner” can include a pet caretaker, pet caregiver, a researcher, a veterinarian, a veterinary technician, and/or another party.

As used herein, a “training data set” can include one or more images or videos and associated data to train a machine-learning model. Each training data set can comprise a training image of one or more data and a corresponding output associated with the image. A training data set can include one or more images or videos of oral cavities of pets. A training data set can be collected via one or more client devices (e.g., crowd-sourced) or collected from other sources (e.g., a database). In certain non-limiting embodiments, the training data set for a dental assessment of a pet can include data from both a treatment group and a control group.

Certain non-limiting embodiments are described below with reference to block diagrams and operational illustrations of methods, processes, devices, and apparatus. It is understood that each block of the block diagrams or operational illustrations, and combinations of blocks in the block diagrams or operational illustrations, can be implemented by means of analog or digital hardware and computer program instructions. These computer program instructions can be provided to a processor of a general-purpose computer to alter its function as detailed herein, a special purpose computer, ASIC, or other programmable data processing apparatus, such that the instructions, which execute via the processor of the computer or other programmable data processing apparatus, implement the functions/acts specified in the block diagrams or operational block or blocks. In some alternate implementations, the functions/acts noted in the blocks can occur out of the order noted in the operational illustrations. For example, two blocks shown in succession can in fact be executed substantially concurrently or the blocks can sometimes be executed in the reverse order, depending upon the functionality/acts involved.

These computer program instructions can be provided to a processor of: a general purpose computer to alter its function to a special purpose; a special purpose computer; ASIC; or other programmable digital data processing apparatus, such that the instructions, which execute via the processor of the computer or other programmable data processing apparatus, implement the functions/acts specified in the block diagrams or operational block or blocks, thereby transforming their functionality in accordance with embodiments herein.

In some non-limiting embodiments, a computer readable medium (or computer-readable storage medium/media) stores computer data, which data can include computer program code (or computer-executable instructions) that is executable by a computer, in machine readable form. By way of example, and not limitation, a computer readable medium can comprise computer readable storage media, for tangible or fixed storage of data, or communication media for transient interpretation of code-containing signals. Computer readable storage media, as used herein, refers to physical or tangible storage (as opposed to signals) and includes without limitation volatile and non-volatile, removable and non-removable media implemented in any method or technology for the tangible storage of information such as computer-readable instructions, data structures, program modules or other data. Computer readable storage media includes, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other physical or material medium which can be used to tangibly store the desired information or data or instructions and which can be accessed by a computer or processor.

Unlike humans, animals should be under general anesthesia for dental X-rays. It is also difficult to examine the teeth of animals because of the muzzle and arrangement of teeth. For humans, dentists can look for markers to understand/identify teeth with issues. However, for animals, this is a harder problem given the different shapes of breed muzzles and markers are not necessarily established.

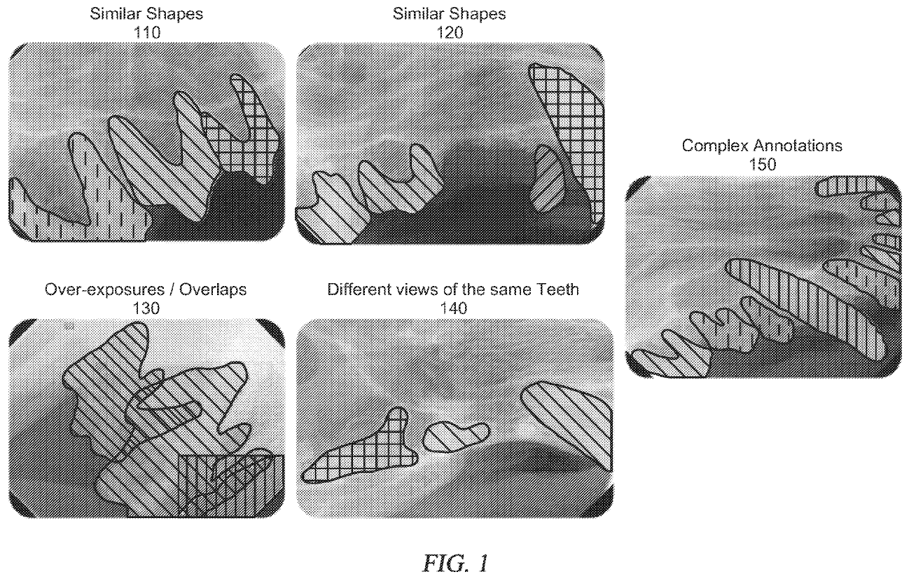

FIG. 1 illustrates example challenges faced with using artificial intelligence (AI) for pet dentistry. As can be seen, these challenges include, but not limited to, similar shapes 110 among the teeth, missing tooth 120, over-exposures or overlaps 130, different views of the same teeth 140, and complex annotations 150.

After a first X-Ray is taken, dentists may realize, after some time, that additional images (e.g., extra films with different positioning or angles) are needed due to the above-referenced challenges. In these cases, an extra round of anesthesia for the animal may be needed to capture the additional images. Additional rounds of anesthesia may increase certain health risks for the animal. Systems and methods consistent with the present disclosure may enable a faster analysis of the dental X-Ray films, reducing considerably the necessity of extra round of general anesthesia. This approach reduces risks associated with multiple rounds of general anesthesia, improves the efficiency on delivering the final diagnostic, and creates a better experience for thousands of animals and their owners per month.

High-quality training data is beneficial for training a robust machine-learning model. Therefore, data may be collected and used for training a machine-learning model for detecting dental pathology. In particular embodiments, data collection may be organized to support three distinct sub-tasks that the machine-learning model needs to perform. One sub-task may be quadrant and view classification, in which the machine-learning model determines the mouth region that the image belongs to and if it is a lateral or occlusal view. Another sub-task may be tooth detection, in which the machine-learning model localizes and provides the coordinates of all the teeth on the image. Another sub-task may be tooth identification, in which the machine-learning model specifies the tooth numbering for the detected teeth according to the Triadan system. The Triadan system provides a consistent method of numbering teeth across different animal species. The first digit of the modified Triadan system denotes the quadrant. The second and third digits denote the tooth position within the quadrant, with the sequence always starting at the midline. Another sub-task may be disease detection, in which the machine-learning model detects pathology on each of the identified teeth. Data sampling and annotations for sub-tasks may be performed independently.

To collect the quadrant and view classification data, a certified veterinary dentist evaluated the images and annotated the quadrant and X-Ray view (lateral or occlusal) for 2511 images. The images were classified on 14 possible classes of full mouth radiographs according to different film position and beam angle. The exact breakdown of the data is referenced in Table 1.

TABLE 1
Radiograph quadrant and view classification data.

| Quadrant | Category | Images | % of dataset |
|---|---|---|---|
| (100-200) | Occlusal view maxillary incisors | 280/2511 | 11.1% |
| (300-400) | Occlusal view mandibular incisors | 248/2511 | 9.9% |
| (100) | Lateral view maxillary caudal premolars/molars | 187/2511 | 7.4% |
| (100) | Lateral view maxillary canine | 140/2511 | 5.6% |
| (100) | Lateral view maxillary rostral premolars | 196/2511 | 7.8% |
| (200) | Lateral view maxillary rostral premolars | 177/2511 | 7.0% |
| (200) | Lateral view maxillary caudal premolars/molars | 184/2511 | 7.3% |
| (200) | Lateral view maxillary canine | 145/2511 | 5.8% |
| (300) | Lateral view mandibular canine | 56/2511 | 2.2% |
| (300) | Lateral view mandibular rostral premolars − bisecting angle | 156/2511 | 6.2% |
| (300) | Lateral view mandibular caudal premolars/molars − parallel | 262/2511 | 10.4% |
| (400) | Lateral view mandibular rostral premolars − bisecting angle | 151/2511 | 6.0% |
| (400) | Lateral view mandibular canine | 85/2511 | 3.4% |
| (400) | Lateral view mandibular caudal premolars/molars − parallel | 244/2511 | 9.7% |

The images were extracted from DICOM files and downsized to 948×676 pixels as it represents a good balance for feature representation and graphics processing unit (GPU) memory. The images comprise data from different sources, including 48815 images from a first source and 9086 images from a second source. Besides annotated images for quadrant detection and view classification, there are manually annotated images for bone loss detection. Systems and methods consistent with embodiments of the present disclosure may use natural language processing (NLP) to extract from dental reports and using them to bring more data.

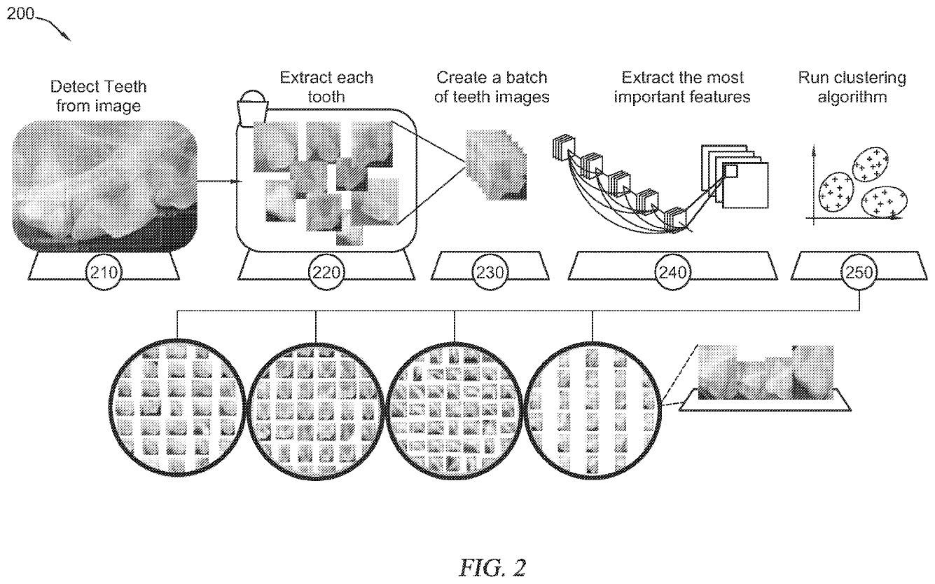

FIG. 2 illustrates an example clustering pipeline 200 for increasing the size of the tooth identification data in accordance with embodiments of the present disclosure. After images are collected and annotated, clustering may be performed on these images to increase the size of the dataset if the manually annotated dataset is too small. As an example, and not by way of limitation, the clustering may comprise generating image embeddings (features) and performing principal component analysis (PCA) and using K-Means (a clustering algorithm) for clustering. As illustrated in FIG. 2, a computing system can detect teeth from an image at step 210. At step 220, the computing system can extract each tooth that has been detected. At step 230, the computing system can create a batch of teeth images. At step 240, the computing system can extract the most important features from the batch of teeth images. At step 250, the computing system can run the clustering algorithm. Although disclosure describes clustering tooth identification data to increase their size, this disclosure contemplates clustering any suitable data such as tooth detection data, disease detection data, and data from dental reports to increase their size.

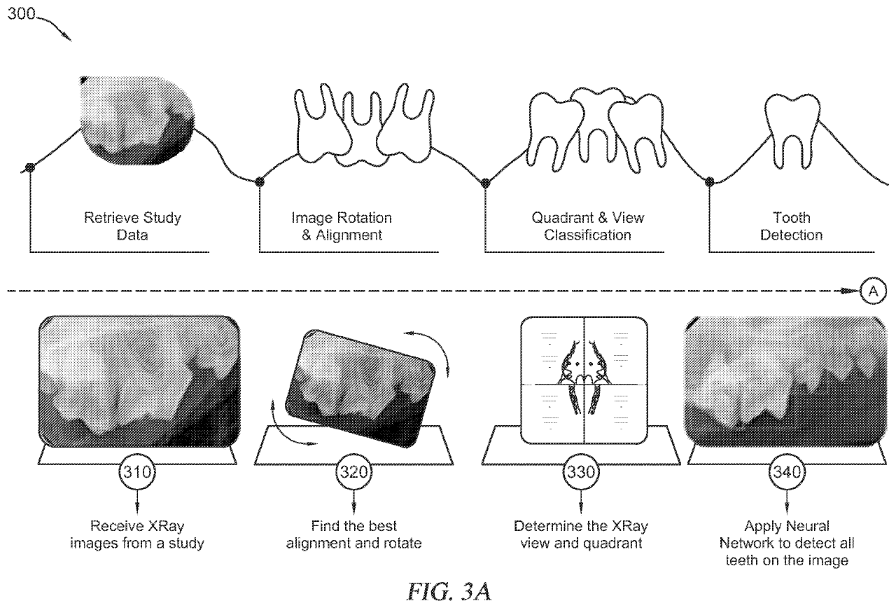

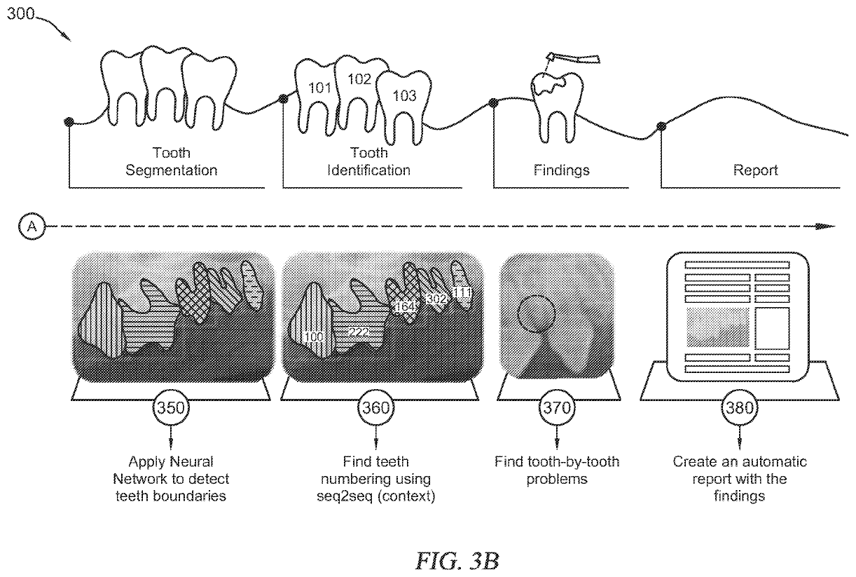

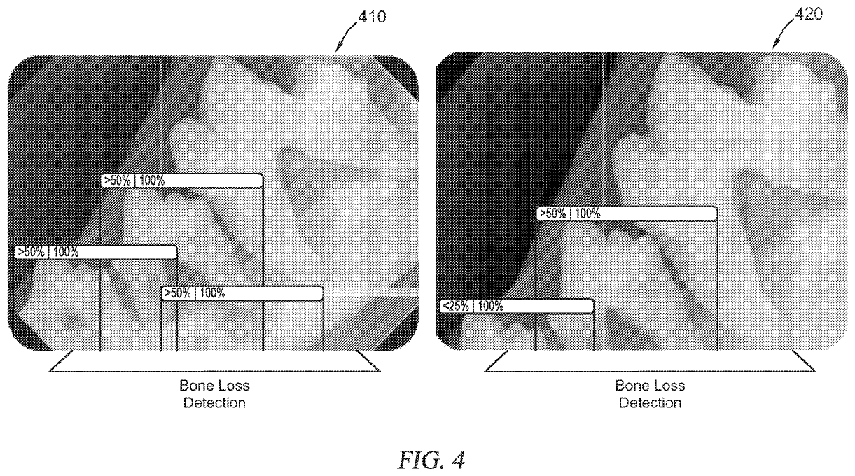

FIGS. 3A-3B illustrate an example flow diagram 300 for detecting pet dental pathology. As illustrated in FIG. 3A, at step 310, the computing system can retrieve study data, e.g., X-ray images from a study. The computing system can perform some image pre-processing at this step. In particular embodiments, and not by way of limitation, the pre-processing can include removing non-dental images. In particular embodiments, the pre-processing can include detecting and removing non-diagnostic images. The computing system can train machine-learning models for detecting non-diagnostic images based on training data comprising both diagnostic images and non-diagnostic images. The computing system may then use such trained machine-learning models to detect non-diagnostic images. In particular embodiments, non-diagnostic images may not include important structures for effectively detecting dental pathology. As an example, and not by way of limitation, if the computing system can't identify what quadrant the X-ray image is from (e.g., top or bottom), such X-ray image can be determined as non-diagnostic. FIG. 4 illustrates an example comparison between a diagnostic image and a non-diagnostic image. As can be seen, image 410 depicts a gum line indicating this is an image of the bottom portion of the mouth, which can be important for detecting bone loss. Therefore, image 410 can be determined as a diagnostic image. However, image 420 does not depict the gum line, so the computing system cannot determine if the image is associated with the top or bottom portion of the mouth. As a result, image 420 can be detected as non-diagnostic.

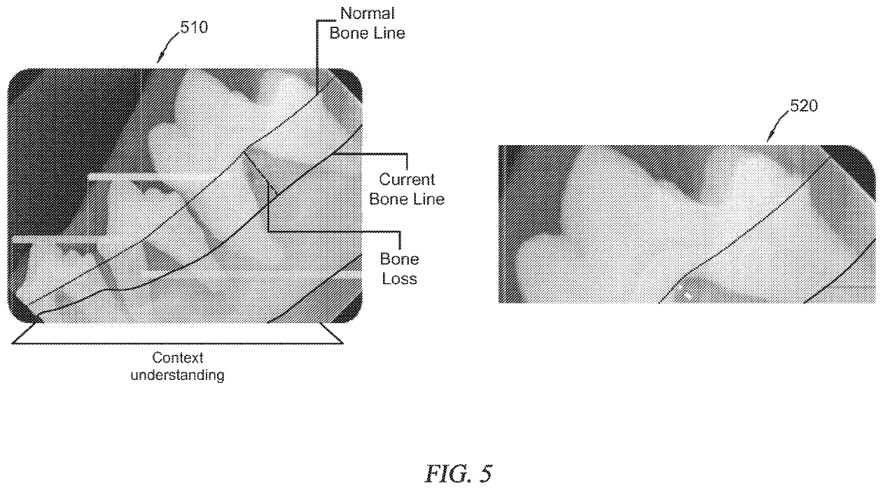

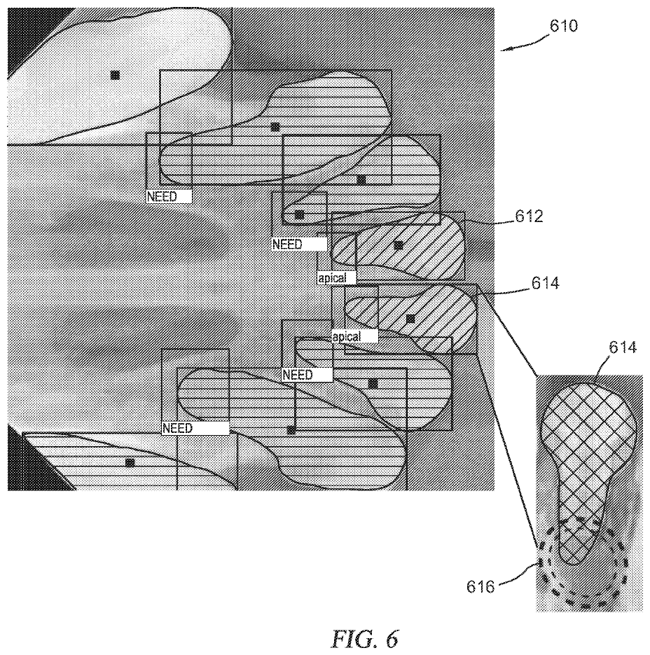

As another example, and not by way of limitation, if the X-ray image does not comprise structures that are the basis of the findings, such X-ray image can be determined as non-diagnostic. FIG. 5 illustrates another example comparison between a diagnostic image and a non-diagnostic image. As can be seen, image 510 depicts a bone line, which can be important to estimate the bone loss. Therefore, image 510 can be determined as a diagnostic image. By contrast, image 520 only covers the crown so the computing system has no basis to understand the bone loss. Thus, image 520 can be determined as a non-diagnostic image. FIG. 6 illustrates an example diagnostic image for apical periodontitis prediction. Image 610 shows a full image of teeth with the area surrounding the root of a tooth, e.g., tooth 612 and tooth 614. To detect apical periodontitis, the computing system may need to analyze the root area 616. Since image 610 comprises such important structure, it can be determined as a diagnostic image. However, if an X-ray image doesn't comprise the root area as exemplified in image 610, such image is considered non-diagnostic.

Referring back to FIG. 3A, at step 320, the computing system can perform image rotation and alignment using a rotation model. In other words, the computing system can find the best alignment and rotate the image accordingly. In particular embodiments, the computing system can rotate the image by 0 degrees (which means the image is well aligned), +/−90 degree, or 180 degrees according to certain standard of certain imaging machines. If the X-ray image is off by a degree that is not of these four angles, the computing system can approximate the image to the target angle since when training the machine-learning models for pathology detection, the images can be forced to be read at a certain measure. In particular embodiments, if an X-ray image is off by a degree between 0 and 90, the computing system can get reliable accuracy for pathology detection. Although this disclosure describes rotating particular images by particular degrees, this disclosure contemplates rotating any suitable image by any other suitable degrees, e.g., +/−10 degrees, +/−45 degrees, etc.

At step 330, the computing system can perform quadrant and view classification. In other words, the computing system can determine the X-ray view and quadrant. Determining the quadrant can be important for reducing the complexity of the machine-learning models on the next stages. The teeth representation depends on the image view, which means the same tooth can look different depending on the beam angle and film position. Providing extra information regarding the quadrant and view can help increase the detection robustness once the machine-learning models are trained to a specific task rather than including all the views together. It can reduce the model complexity and enable model re-usability due to the existent symmetry on left and right part of the mouth. Moreover, the quadrant and view information can be relevant to the clinical analysis so providing a detailed context about the radiography and teeth localization can help the dentist interpret the model results. Due to the high granularity of the annotated data, similar image views can be combined together to increase the amount of data per class and reduce the total number of classes that led to accuracy increase. Table 2 lists example combined quadrant and view training data.

TABLE 2
Combined quadrant and view training data.

| Annotation Class | Training Class |
|---|---|
| (100-200) Occlusal view maxillary incisors | 100-200 (Occlusal) |
| (300-400) Occlusal view mandibular incisors | 300-400 (Occlusal) |
| (100) Lateral view maxillary caudal premolars molars + | 100 (Lateral) |
| (100) Lateral view maxillary rostral premolars + | |
| (100) Lateral view maxillary canine | |
| (200) Lateral view maxillary caudal premolars molars + | 200 (Lateral) |
| (200) Lateral view maxillary rostral premolars + | |
| (200) Lateral view maxillary canine | |
| (300) Lateral view mandibular caudal premolars molars parallel + | 300 (Lateral) |
| (300) Lateral view mandibular rostral premolars bisecting angle + | |
| (300) Lateral view mandibular canine optional | |
| (400) Lateral view mandibular caudal premolars molars parallel + | 400 (Lateral) |
| (400) Lateral view mandibular rostral premolars bisecting angle + | |
| (400) Lateral view mandibular canine optional | |

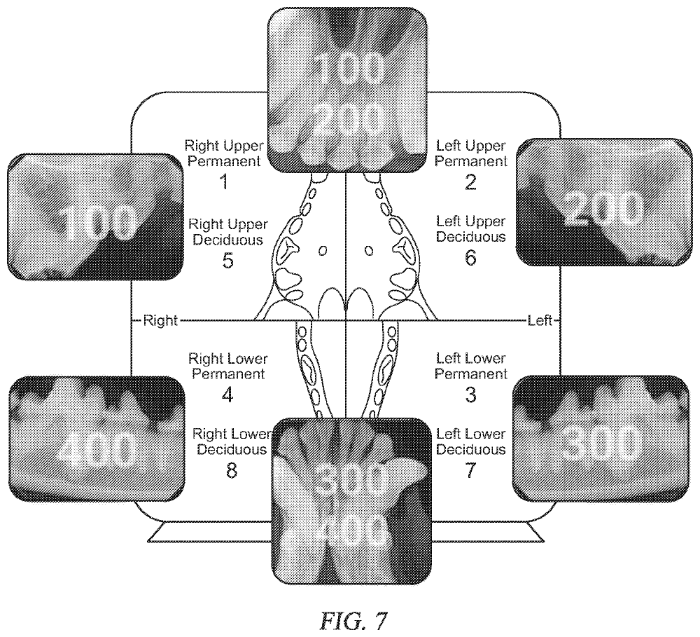

In particular embodiments, a deep-learning model can be trained by fine-tuning a pre-trained weight that determines the quadrant and the view of a particular X-ray image as described on the Triadan system. As an example, and not by way of limitation, the deep-learning model can be based on a Resnet101 architecture using the dataset described in Table 1 with the 6 combined classes described in Table 2. As another example and not by way of limitation, the pre-trained weight can be determined based on an ImageNet dataset. In particular embodiments, the deep-learning model can be trained using ADAM optimizer and cross entropy loss with learning rate of 3e^{-4.} Combining the classes as aforementioned can boost the classification, reaching around F1 score of 96%. FIG. 7 illustrates example quadrants based on the Triadan system. The quadrants are determined by numbers from 1 to 4 as the first element of the Triadan system. The views are determined based on whether there is a composition of quadrants or not. For instance, if the model result is 1, it is a lateral view, and the image is from the first quadrant. However, if the model result is 1-2, it is an occlusal view, and the image has parts on the first and second quadrants.

Referring back to FIG. 3A, the computing system can perform tooth detection at step 340. In particular embodiments, the computing system can process images by applying a deep-learning model, e.g., neural network, to detect all teeth on the image. As an example and not by way of limitation, the deep-learning model can be based on a Faster-RCNN architecture with a Resnet101 backend that may be trained using a pre-trained weight. The pre-trained weight can be determined based on the ImageNet dataset. The deep-learning model can determine the boxes coordinates for all the possible teeth on an image and provide a score with probabilities of the detections being a tooth.

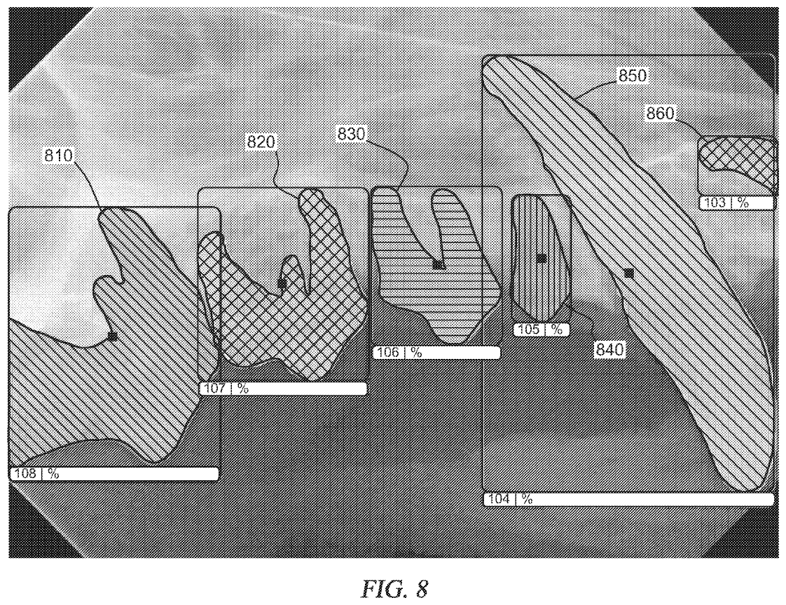

At step 350 in FIG. 3B, the computing system can perform tooth segmentation. In particular embodiments, the tooth detection can use an extra model for applying instance segmentation, e.g., neural network, to detect teeth boundaries for the detected teeth. As an example, and not by way of limitation, this model can be a deep-learning model, which can be based on MaskRCNN architecture that uses a ResNet101 architecture as a backend. In particular embodiments, the deep-learning model can be trained using a pre-trained weight. As an example, and not by way of limitation, the pre-trained weight can be determined based on the ImageNet dataset. Given the X-Ray image, the model can determine the same information as the tooth detection phase, but with the addition of true tooth boundaries and the masked tooth without the background. FIG. 8 illustrates an example tooth segmentation. As can be seen, the computing system can segment the teeth by generating boundaries (boundary 810, boundary 820, boundary 830, boundary 840, boundary 850, and boundary 860) for each tooth. There are additionally bounding boxes, within each of them each tooth resides.

At step 360 in FIG. 3B, the computing system can perform tooth identification by finding teeth numbering using, e.g., a sequence-to-sequence (seq2seq) model taking into account of context. After determining the teeth on the image, the computing system may address the tooth identification, which can comprise numbering teeth according to the Triadan system, e.g., for cats and dogs. In particular embodiments, a series of deep-learning models may be based on transformers architecture with a Resnet50 backend and DETR framework and train them using a pre-trained Resnet weight for tooth identification. As an example, and not by way of limitation, the pre-trained Resnet weight can be determined based on the ImageNet dataset. In particular embodiments, based on these models, the computing system can learn to use the global context to help with tooth numbering, simulating the way humans do when they are analyzing radiographies. It can dramatically increase the result quality, especially in cases like missing teeth, baby teeth and some other abnormalities. Because of the mouth symmetry, there can be two models for tooth identification, one for the top (maxilla) and other for the (mandible), since the teeth characteristics vary depending on the part of the mouth they belong to.

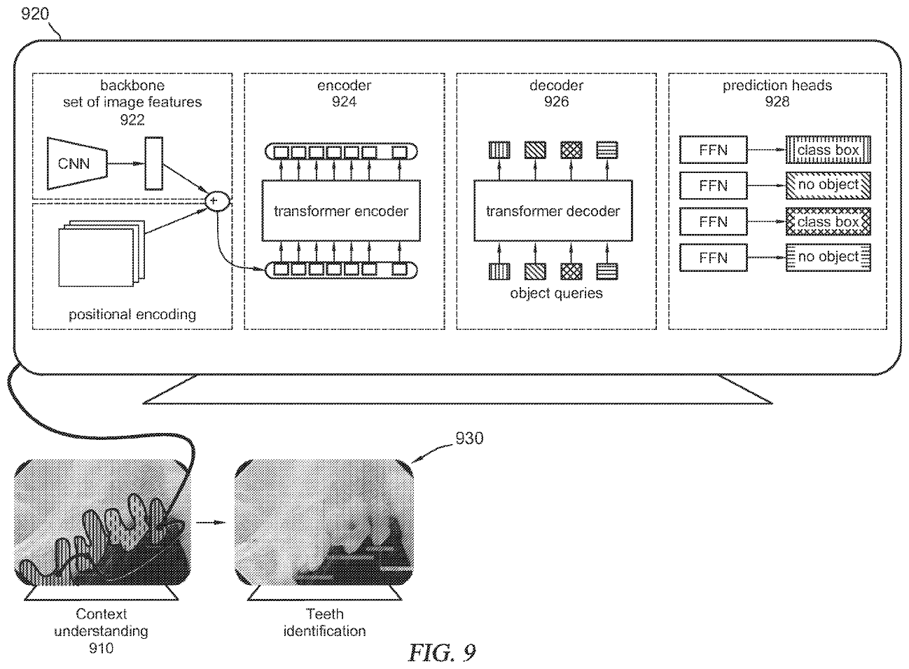

FIG. 9 illustrates an example teeth identification taking into account of context understanding. In particular embodiments, context understanding 910 can comprise getting more information about the context associated with each tooth. As an example, and not by way of limitation, the context can include relative sizes of the teeth. The context can be fed into the deep-learning model 920 together with the X-ray image 930 for teeth identification. The backbone 922 can generate a set of image features for the context based on one or more convolutional neural networks (CNN) and positional encoding (i.e., encoding the positional information of each tooth). The output from the backbone 922 may then be processed by an encoder 924, e.g., a transformer encoder. The output from the encoder 924 may then be processed by a decoder 926, e.g., a transformer decoder, based on object queries. The output from the decoder 926 may then be used to determine prediction heads 928. As an example, and not by way of limitation, prediction heads 928 can be determined based on a plurality of feed-forward neural networks (FFN), with each outputting a class box or “no object”. With the context understanding, the deep-learning model can more effectively accomplish teeth identification.

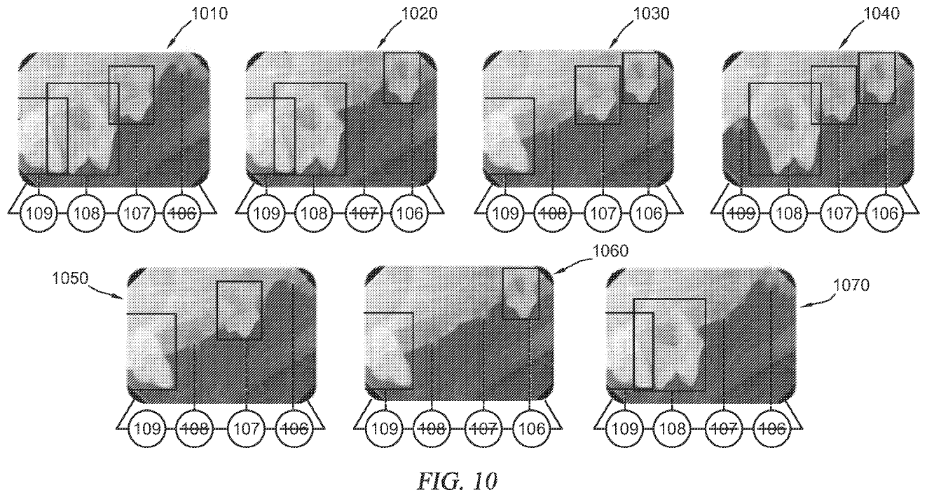

FIG. 10 illustrates an example test experiment to evaluate teeth identification. FIG. 10 shows X-ray images, where one or more teeth were synthetically removed to prove the model capability on determining the teeth identification even with missing tooth/teeth. For example, image 1010 shows the model can effectively identify the missing tooth 106. As another example, image 1020 shows the model can effectively identify the missing tooth 107. As another example, image 1030 shows the model can effectively identify the missing tooth 108. As another example, image 1040 shows the model can effectively identify the missing tooth 109. As another example, image 1050 shows the model can effectively identify the missing teeth 106 and 108. As another example, image 1060 shows the model can effectively identify the missing teeth 107 and 108. As another example, image 1070 shows the model can effectively identify the missing teeth 106 and 107.

Referring back to FIG. 3B again, the computing system can then determine findings at step 370. Specifically, the computing system can find tooth-by-tooth problems. In particular embodiments, the computing system can determine if a tooth is healthy or if it has any pathology. In particular embodiments, a series of deep-learning models may be based on transformers architecture with a Resnet50 backend and DETR framework and train them using a pre-trained Resnet weight for tooth pathology detection. As an example, and not by way of limitation, the pre-trained Resnet weight can be determined based on the ImageNet dataset. In particular embodiments, the computing system can determine different modalities of pathology. As an example and not by way of limitation, the modalities comprise one or more of an endodontic disease, a periodontal disease, tooth resorption, tooth fracture, or any other suitable dental disease.

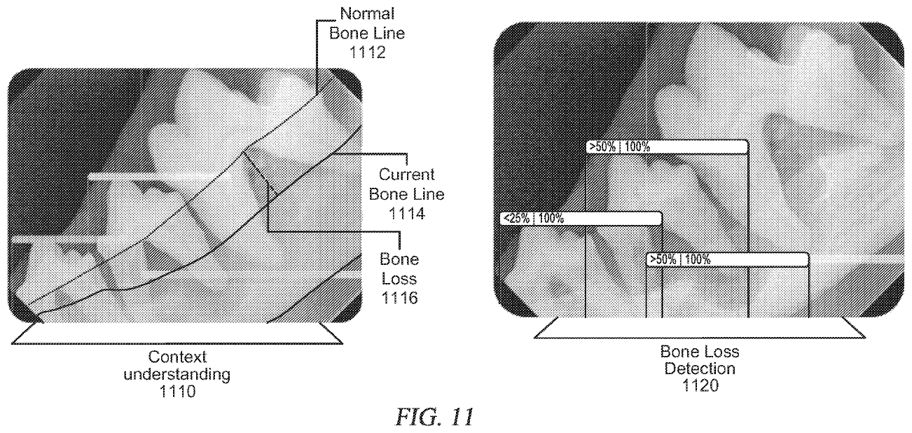

For periodontal disease, bone loss detection is one use case. Because of the mouth symmetry, there can be two models for bone loss detection, one for the top (maxilla) and the other for the (mandible), because the teeth characteristics vary depending on the part of the mouth they belong to. In particular embodiments, the models can determine a plurality of levels of bone loss. As an example, and not by way of limitation, the levels can comprise <25%, 25-50%, >50% and no evidence of periodontal disease. In particular embodiments, context understanding can be similarly used to improve bone loss detection. FIG. 11 illustrates an example context understanding for bone loss detection. For context understanding 1110, the computing system can identify the normal bone line 1112 and current bone line 1114, which can help determine bone loss 1116. As illustrated in bone loss detection 1120 in FIG. 11, the computing system can further determine different levels of bone loss, e.g., <25% for the left tooth 1122 and >50% for both the middle tooth 1124 and the right tooth 1126.

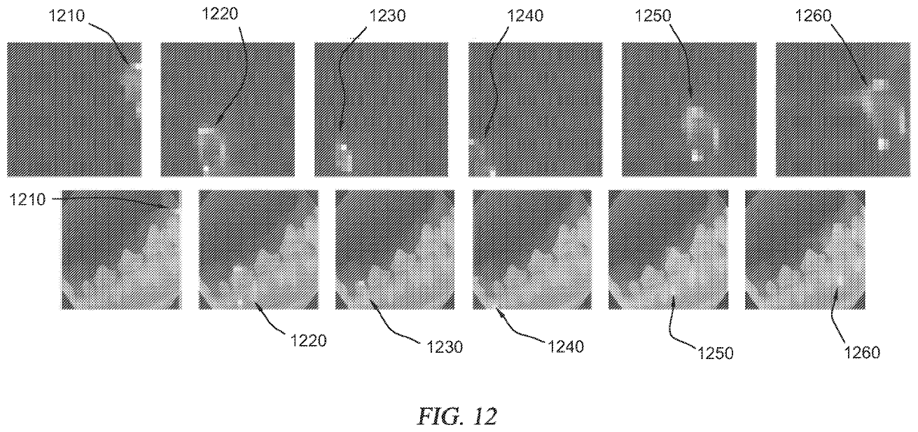

In particular embodiments, the computing system can provide model interpretability of the one or more machine-learning models used for dental pathology detection. FIG. 12 illustrates example decoder attention maps. The first row shows the decoder attention maps whereas the bottom row shows the corresponding X-ray images, respectively. The decoder attention maps show the top activations of the models. FIG. 12 illustrates the output of the decoder stage of the deep-learning model disclosed herein. In FIG. 12, the most important regions (region 1210, region 1220, region 1230, region 1240, region 1250, and region 1260) are summarized (and highlighted) in the image that led the deep-learning model to the result. FIG. 12 demonstrates the ability of the deep-learning model to determine the most important features that represents each tooth.

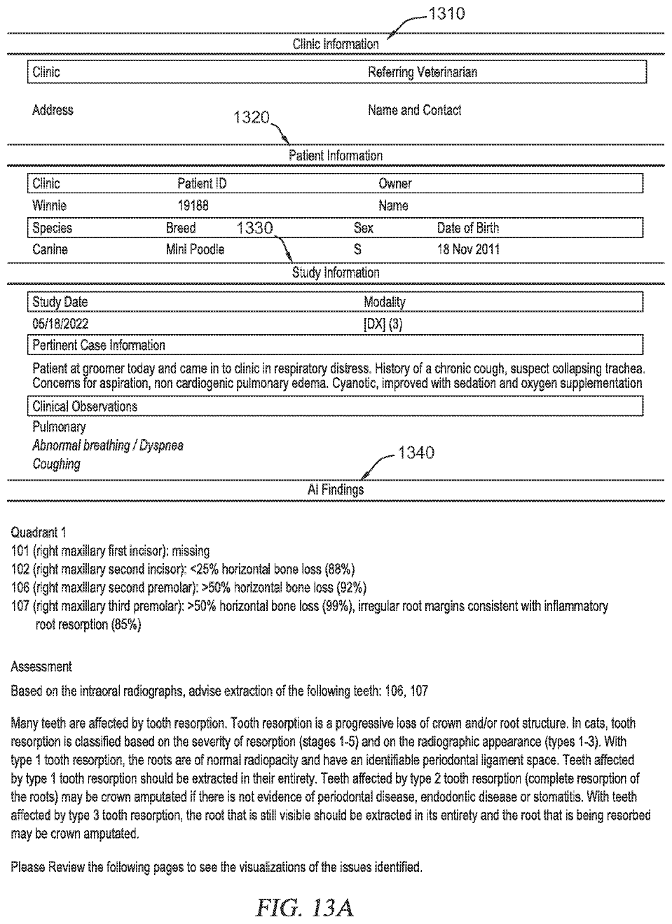

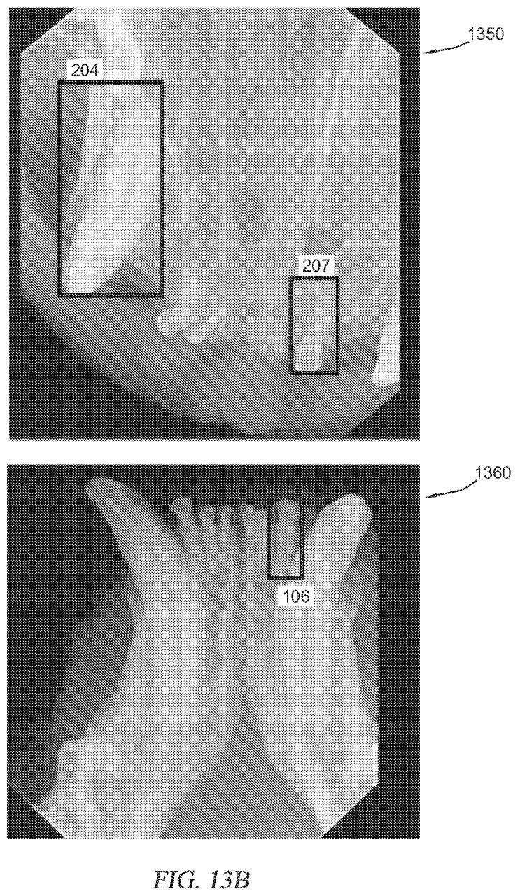

Referring back again to FIG. 3B, the computing system can further create an automatic report with the findings at step 380. The report can comprise the information from the models along with the tooth identification, quadrant, finding and grade level of the tooth problem. FIGS. 13A-13B illustrate an example report. FIG. 13A illustrates textual descriptions of the report. The textual descriptions include clinic information 1310, patient information 1320, study information 1330 and AI findings 1340. For example, the patient information 1320 includes the species, breed, sex, and date of birth for the animal. As another example, the AI finds 1340 show that for Quadrant 1, tooth 101 (right maxillary first incisor) is missing, tooth 102 (right maxillary second incisor) has <25% horizontal bone loss with a probability of 88%, tooth 106 (right maxillary second premolar) has >50% horizontal bone loss with a probability of 92%, and tooth 107 (right maxillary third premolar) has >50% horizontal bone loss with a probability of 99% and has irregular root margins consistent with inflammatory root resorption with a probability of 85%. The AI findings 1340 further include assessment indicating that based on the intraoral radiographs, it is advised to extract the teeth 106 and 107. FIG. 13B illustrates example evaluated images. In particular embodiments, the report can further include the evaluated images of the teeth. As can be seen, the top image 1350 is focused on teeth 204 and 207 whereas the bottom image 1360 is focused on tooth 106.

In particular embodiments, dental pathology detection can be performed in a cloud computing system. The cloud computing system can detect dental pathology for multiple studies, each comprising multiple X-ray images (e.g., 30-40 images). In particular embodiments, all the images can be processed in parallel. As previously described, detecting tooth pathology can be based on multiple machine-learning models, e.g., a rotation model, a tooth detection model, a tooth numbering model, etc. In particular embodiments, each image can be processed by these multiple machine-learning models in parallel. In particular embodiments, the cloud computing system may wait for all images associated with the entire mouth to be processed before a report can be generated. In particular embodiments, to aggregate the processed data, the cloud computing system can utilize different confidence measures. The cloud computing system can determine how many images remain to be processed and when the processing of all the images is completed.

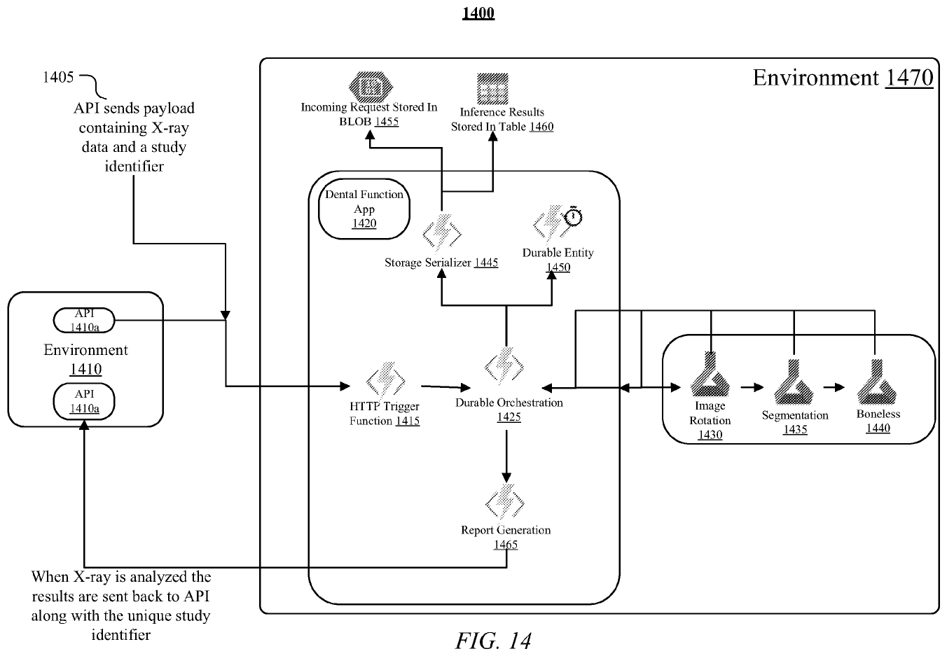

FIG. 14 illustrates an example flow diagram 1400 for parallel processing of multiple dental X-ray images in a cloud computing system. At step 1405, an API can send a payload (e.g., JSON) comprising X-ray data and a study identifier. A call can be initiated when a vet requests a review (e.g., an authorization token). An API 1410a associated with the environment 1410 for dental pathology detection can then send the payload to an HTTP trigger function 1415, which is part of a dental function a app 1420. The HTTP trigger function 1415 can call a durable orchestration module 1425 of the dental function app 1420. The durable orchestration module 1425 can then call different models such as image rotation 1430, segmentation 1435, and bone loss detection 1440. The results from these models can be returned to the durable orchestration module 1425. The durable orchestration module 1425 can then send the results to a storage serializer 1445 and a durable entity 1450. The serializer 1445 can create an incoming request stored in blob 1455 and inference results stored in table 1460. In particular embodiments, the durable orchestration module 1425 can perform report generation 1465 based on the returned results from the models. When the X-ray data is analyzed, the results can be sent back to the API 1410a associated with the environment 1410 along with the unique study identifier (e.g., the authorization token). As illustrated in FIG. 14, the dental function app 1420, the models for image rotation 1430, segmentation 1435, and bone loss detection 1440, the BLOB 1455, and the table 1460 can be hosted in the environment 1470 designated for dental pathology detection of multiple X-ray images from multiple studies.

As previously described, the cloud computing system can parallel the processing of multiple X-ray images using multiple models. In particular embodiments, the durable orchestration module 1425 can generate logic to enable such parallel processing. As an example, and not by way of limitation, the logic can be based on one or more finite state machines. In particular embodiments, the cloud computing system can train the logic on what to do once receiving the images to create the steps for parallel processing. As an example, and not by way of limitation, the durable orchestration module 1425 can set a timer, e.g., 15 minutes. After detecting no more images to process, the durable orchestration module 1425 can wait for 15 minutes before generating the report. Once an image is received, the durable orchestration module 1420 can start a timer and determine a timeout so the cloud computing system doesn't wait for additional images forever. For example, if the cloud computing system received 15 images (as compared to 30 to 40 images commonly in a study), the cloud computing system can send a partial report based on the 15 images upon determining a timeout of 15 minutes. As another example, and not by way of limitation, the logic can comprise one or more “if . . . else . . . ” commands. Based on the logic, the cloud computing system can effectively combine the processing of all X-ray images in a parallel manner.

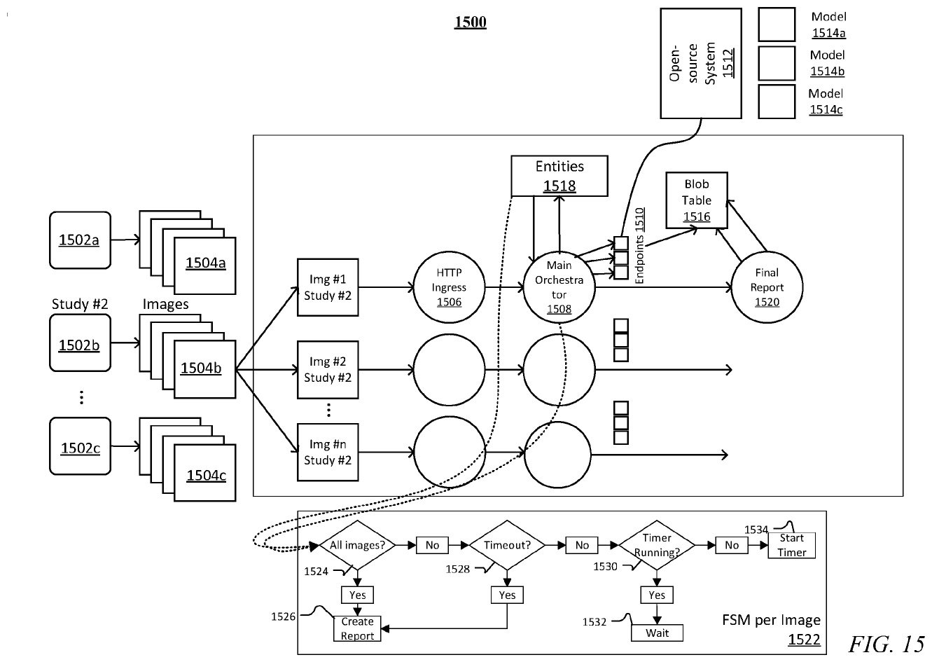

FIG. 15 illustrates an example an example flow diagram 1500 for stateful orchestration. In particular embodiments, the cloud computing system may need confirmation from all studies. The cloud computing system can detect when new images come and link them to the first set of received images. The cloud computing system can further download all images to prepare a full report. In particular embodiments, logic can be programmed to account for all of the aforementioned steps. As illustrated in FIG. 15, the cloud computing system can receive requests for dental pathology detection of multiple studies 1502a-1502c, each comprising multiple X-ray images 1504a-1504c. As an example, and not by way of limitation, study #2 (1502b) can comprise images 1504b. Image #1 of study #2 can be received at HTTP ingress 1506. The HTTP ingress 1506 can then call the main orchestrator 1508. The main orchestrator 1508 can access, via multiple endpoints 1510, an open-source system 1512, which automates deployment, scaling, and management of multiple models 1514a-1514c. With these models 1514a-1514c, image #1 of study #2 can be processed. The main orchestrator 1508 can store the processing results in the blob table 1516. In particular embodiments, the main orchestrator 1508 can communicate with entities 1518 comprising information associated with remaining images, deadline, timer flag, etc. If there are no remaining images (i.e., all X-ray images are processed), final report 1520 can be generated based on the blob table 1516.

In particular embodiments, either the main orchestrator 1508 or the entities 1518 can access the finite state machine per image 1522. Within the finite state machine 1522, at step 1524, the logic instructs determining whether all images have been processed. If all the images are processed, the logic instructs creating report at step 1526. If not all images are processed, the logic instructs determining if the timeout (e.g., 15 minutes) is reached at step 1528. If the timeout is reached, the logic instructs creating report at step 1526. If the timeout is not reached, the logic instructs checking whether the timer is running at step 1530. If the timer is running, the logic instructs waiting for additional images at step 1532. If the timer is not running, the logic instructs starting a timer at step 1534.

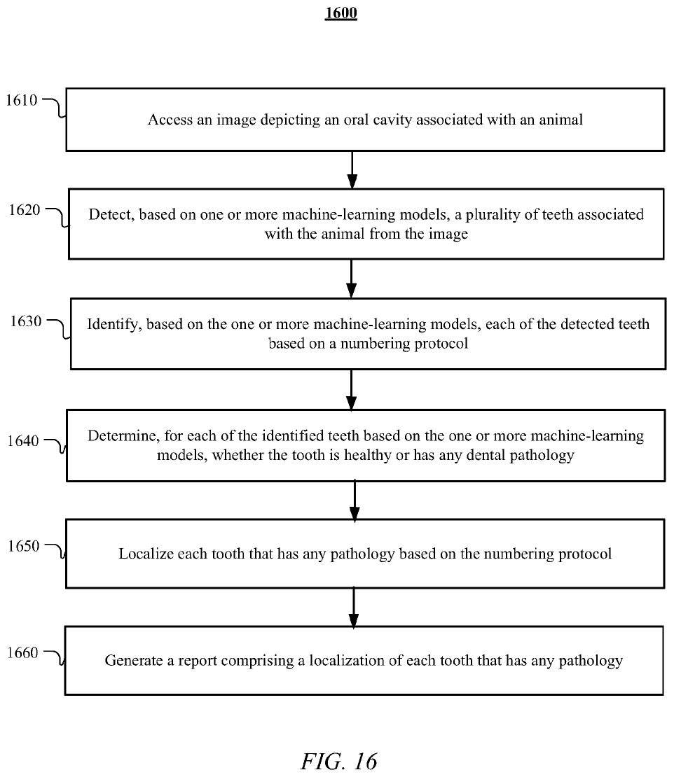

FIG. 16 illustrates an example method 1600 for detecting tooth pathology. The method may begin at step 1610, where the computing system may access a first image depicting an oral cavity associated with an animal. At step 1620, the computing system may detect, based on one or more machine-learning models, a plurality of teeth associated with the animal from the first image. At step 1630, the computing system may identify, based on the one or more machine-learning models, each of the detected teeth based on a numbering protocol. At step 1640, the computing system may determine, for each of the identified teeth based on the one or more machine-learning models, whether the tooth is healthy or has any dental pathology. At step 1650, the computing system may localize each tooth that has any pathology based on the numbering protocol. At step 1660, the computing system may generate a first report comprising a localization of each tooth that has any pathology. Particular embodiments may repeat one or more steps of the method of FIG. 16, where appropriate. Although this disclosure describes and illustrates particular steps of the method of FIG. 16 as occurring in a particular order, this disclosure contemplates any suitable steps of the method of FIG. 16 occurring in any suitable order. Moreover, although this disclosure describes and illustrates an example method for detecting tooth pathology including the particular steps of the method of FIG. 16, this disclosure contemplates any suitable method for detecting tooth pathology including any suitable steps, which may include all, some, or none of the steps of the method of FIG. 16, where appropriate. Furthermore, although this disclosure describes and illustrates particular components, devices, or systems carrying out particular steps of the method of FIG. 16, this disclosure contemplates any suitable combination of any suitable components, devices, or systems carrying out any suitable steps of the method of FIG. 16.

For the purposes of this disclosure the term “user”, “subscriber” “consumer” or “customer” should be understood to refer to a user of an application or applications as described herein and/or a consumer of data supplied by a data provider. By way of example, and not limitation, the term “user” or “subscriber” can refer to a person who receives data provided by the data or service provider over the Internet in a browser session, or can refer to an automated software application which receives the data and stores or processes the data.

Those skilled in the art will recognize that the methods and systems of the present disclosure can be implemented in many manners and as such are not to be limited by the foregoing exemplary embodiments and examples. In other words, functional elements being performed by single or multiple components, in various combinations of hardware and software or firmware, and individual functions, can be distributed among software applications at either the client level or server level or both. In this regard, any number of the features of the different embodiments described herein can be combined into single or multiple embodiments, and alternate embodiments having fewer than, or more than, all of the features described herein are possible.

Functionality can also be, in whole or in part, distributed among multiple components, in manners now known or to become known. Thus, myriad software/hardware/firmware combinations are possible in achieving the functions, features, interfaces and preferences described herein. Moreover, the scope of the present disclosure covers conventionally known manners for carrying out the described features and functions and interfaces, as well as those variations and modifications that can be made to the hardware or software or firmware components described herein as would be understood by those skilled in the art now and hereafter.

Furthermore, the embodiments of methods presented and described as flowcharts in this disclosure are provided by way of example in order to provide a more complete understanding of the technology. The disclosed methods are not limited to the operations and logical flow presented herein. Alternative embodiments are contemplated in which the order of the various operations is altered and in which sub-operations described as being part of a larger operation are performed independently.

While various embodiments have been described for purposes of this disclosure, such embodiments should not be deemed to limit the teaching of this disclosure to those embodiments. Various changes and modifications can be made to the elements and operations described above to obtain a result that remains within the scope of the systems and processes described in this disclosure.

While the disclosed subject matter is described herein in terms of certain preferred embodiments, those skilled in the art will recognize that various modifications and improvements can be made to the disclosed subject matter without departing from the scope thereof. Moreover, although individual features of one non-limiting embodiment of the disclosed subject matter can be discussed herein or shown in the drawings of the one non-limiting embodiment and not in other embodiments, it should be apparent that individual features of one non-limiting embodiment can be combined with one or more features of another embodiment or features from a plurality of embodiments.

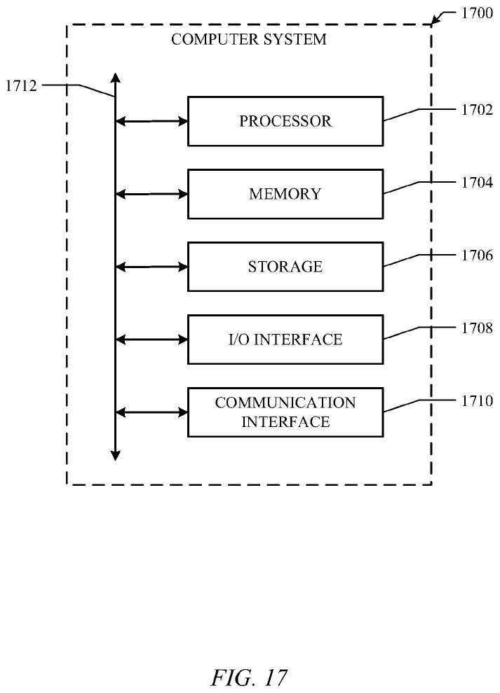

FIG. 17 illustrates an example computer system 1700. In particular embodiments, one or more computer systems 1700 perform one or more steps of one or more methods described or illustrated herein. In particular embodiments, one or more computer systems 1700 provide functionality described or illustrated herein. In particular embodiments, software running on one or more computer systems 1700 performs one or more steps of one or more methods described or illustrated herein or provides functionality described or illustrated herein. Particular embodiments include one or more portions of one or more computer systems 1700. Herein, reference to a computer system may encompass a computing device, and vice versa, where appropriate. Moreover, reference to a computer system may encompass one or more computer systems, where appropriate.

This disclosure contemplates any suitable number of computer systems 1700. This disclosure contemplates computer system 1700 taking any suitable physical form. As example and not by way of limitation, computer system 1700 may be an embedded computer system, a system-on-chip (SOC), a single-board computer system (SBC) (such as, for example, a computer-on-module (COM) or system-on-module (SOM)), a desktop computer system, a laptop or notebook computer system, an interactive kiosk, a mainframe, a mesh of computer systems, a mobile telephone, a personal digital assistant (PDA), a server, a tablet computer system, or a combination of two or more of these. Where appropriate, computer system 1700 may include one or more computer systems 1700; be unitary or distributed; span multiple locations; span multiple machines; span multiple data centers; or reside in a cloud, which may include one or more cloud components in one or more networks. Where appropriate, one or more computer systems 1700 may perform without substantial spatial or temporal limitation one or more steps of one or more methods described or illustrated herein. As an example and not by way of limitation, one or more computer systems 1700 may perform in real time or in batch mode one or more steps of one or more methods described or illustrated herein. One or more computer systems 1700 may perform at different times or at different locations one or more steps of one or more methods described or illustrated herein, where appropriate.

In particular embodiments, computer system 1700 includes a processor 1702, memory 1704, storage 1706, an input/output (I/O) interface 1708, a communication interface 1710, and a bus 1712. Although this disclosure describes and illustrates a particular computer system having a particular number of particular components in a particular arrangement, this disclosure contemplates any suitable computer system having any suitable number of any suitable components in any suitable arrangement.

In particular embodiments, processor 1702 includes hardware for executing instructions, such as those making up a computer program. As an example and not by way of limitation, to execute instructions, processor 1702 may retrieve (or fetch) the instructions from an internal register, an internal cache, memory 1704, or storage 1706; decode and execute them; and then write one or more results to an internal register, an internal cache, memory 1704, or storage 1706. In particular embodiments, processor 1702 may include one or more internal caches for data, instructions, or addresses. This disclosure contemplates processor 1702 including any suitable number of any suitable internal caches, where appropriate. As an example and not by way of limitation, processor 1702 may include one or more instruction caches, one or more data caches, and one or more translation lookaside buffers (TLBs). Instructions in the instruction caches may be copies of instructions in memory 1704 or storage 1706, and the instruction caches may speed up retrieval of those instructions by processor 1702. Data in the data caches may be copies of data in memory 1704 or storage 1706 for instructions executing at processor 1702 to operate on; the results of previous instructions executed at processor 1702 for access by subsequent instructions executing at processor 1702 or for writing to memory 1704 or storage 1706; or other suitable data. The data caches may speed up read or write operations by processor 1702. The TLBs may speed up virtual-address translation for processor 1702. In particular embodiments, processor 1702 may include one or more internal registers for data, instructions, or addresses. This disclosure contemplates processor 1702 including any suitable number of any suitable internal registers, where appropriate. Where appropriate, processor 1702 may include one or more arithmetic logic units (ALUs); be a multi-core processor; or include one or more processors 1702. Although this disclosure describes and illustrates a particular processor, this disclosure contemplates any suitable processor.

In particular embodiments, memory 1704 includes main memory for storing instructions for processor 1702 to execute or data for processor 1702 to operate on. As an example and not by way of limitation, computer system 1700 may load instructions from storage 1706 or another source (such as, for example, another computer system 1700) to memory 1704. Processor 1702 may then load the instructions from memory 1704 to an internal register or internal cache. To execute the instructions, processor 1702 may retrieve the instructions from the internal register or internal cache and decode them. During or after execution of the instructions, processor 1702 may write one or more results (which may be intermediate or final results) to the internal register or internal cache. Processor 1702 may then write one or more of those results to memory 1704. In particular embodiments, processor 1702 executes only instructions in one or more internal registers or internal caches or in memory 1704 (as opposed to storage 1706 or elsewhere) and operates only on data in one or more internal registers or internal caches or in memory 1704 (as opposed to storage 1706 or elsewhere). One or more memory buses (which may each include an address bus and a data bus) may couple processor 1702 to memory 1704. Bus 1712 may include one or more memory buses, as described below. In particular embodiments, one or more memory management units (MMUs) reside between processor 1702 and memory 1704 and facilitate accesses to memory 1704 requested by processor 1702. In particular embodiments, memory 1704 includes random access memory (RAM). This RAM may be volatile memory, where appropriate. Where appropriate, this RAM may be dynamic RAM (DRAM) or static RAM (SRAM). Moreover, where appropriate, this RAM may be single-ported or multi-ported RAM. This disclosure contemplates any suitable RAM. Memory 1704 may include one or more memories 1704, where appropriate. Although this disclosure describes and illustrates particular memory, this disclosure contemplates any suitable memory.

In particular embodiments, storage 1706 includes mass storage for data or instructions. As an example and not by way of limitation, storage 1706 may include a hard disk drive (HDD), a floppy disk drive, flash memory, an optical disc, a magneto-optical disc, magnetic tape, or a Universal Serial Bus (USB) drive or a combination of two or more of these. Storage 1706 may include removable or non-removable (or fixed) media, where appropriate. Storage 1706 may be internal or external to computer system 1700, where appropriate. In particular embodiments, storage 1706 is non-volatile, solid-state memory. In particular embodiments, storage 1706 includes read-only memory (ROM). Where appropriate, this ROM may be mask-programmed ROM, programmable ROM (PROM), erasable PROM (EPROM), electrically erasable PROM (EEPROM), electrically alterable ROM (EAROM), or flash memory or a combination of two or more of these. This disclosure contemplates mass storage 1706 taking any suitable physical form. Storage 1706 may include one or more storage control units facilitating communication between processor 1702 and storage 1706, where appropriate. Where appropriate, storage 1706 may include one or more storages 1706. Although this disclosure describes and illustrates particular storage, this disclosure contemplates any suitable storage.

In particular embodiments, I/O interface 1708 includes hardware, software, or both, providing one or more interfaces for communication between computer system 1700 and one or more I/O devices. Computer system 1700 may include one or more of these I/O devices, where appropriate. One or more of these I/O devices may enable communication between a person and computer system 1700. As an example and not by way of limitation, an I/O device may include a keyboard, keypad, microphone, monitor, mouse, printer, scanner, speaker, still camera, stylus, tablet, touch screen, trackball, video camera, another suitable I/O device or a combination of two or more of these. An I/O device may include one or more sensors. This disclosure contemplates any suitable I/O devices and any suitable I/O interfaces 1708 for them. Where appropriate, I/O interface 1708 may include one or more device or software drivers enabling processor 1702 to drive one or more of these I/O devices. I/O interface 1708 may include one or more I/O interfaces 1708, where appropriate. Although this disclosure describes and illustrates a particular I/O interface, this disclosure contemplates any suitable I/O interface.

In particular embodiments, communication interface 1710 includes hardware, software, or both providing one or more interfaces for communication (such as, for example, packet-based communication) between computer system 1700 and one or more other computer systems 1700 or one or more networks. As an example and not by way of limitation, communication interface 1710 may include a network interface controller (NIC) or network adapter for communicating with an Ethernet or other wire-based network or a wireless NIC (WNIC) or wireless adapter for communicating with a wireless network, such as a WI-FI network. This disclosure contemplates any suitable network and any suitable communication interface 1710 for it. As an example and not by way of limitation, computer system 1700 may communicate with an ad hoc network, a personal area network (PAN), a local area network (LAN), a wide area network (WAN), a metropolitan area network (MAN), or one or more portions of the Internet or a combination of two or more of these. One or more portions of one or more of these networks may be wired or wireless. As an example, computer system 1700 may communicate with a wireless PAN (WPAN) (such as, for example, a BLUETOOTH WPAN), a WI-FI network, a WI-MAX network, a cellular telephone network (such as, for example, a Global System for Mobile Communications (GSM) network), or other suitable wireless network or a combination of two or more of these. Computer system 1700 may include any suitable communication interface 1710 for any of these networks, where appropriate. Communication interface 1710 may include one or more communication interfaces 1710, where appropriate. Although this disclosure describes and illustrates a particular communication interface, this disclosure contemplates any suitable communication interface.

In particular embodiments, bus 1712 includes hardware, software, or both coupling components of computer system 1700 to each other. As an example and not by way of limitation, bus 1712 may include an Accelerated Graphics Port (AGP) or other graphics bus, an Enhanced Industry Standard Architecture (EISA) bus, a front-side bus (FSB), a HYPERTRANSPORT (HT) interconnect, an Industry Standard Architecture (ISA) bus, an INFINIBAND interconnect, a low-pin-count (LPC) bus, a memory bus, a Micro Channel Architecture (MCA) bus, a Peripheral Component Interconnect (PCI) bus, a PCI-Express (PCIe) bus, a serial advanced technology attachment (SATA) bus, a Video Electronics Standards Association local (VLB) bus, or another suitable bus or a combination of two or more of these. Bus 1712 may include one or more buses 1712, where appropriate. Although this disclosure describes and illustrates a particular bus, this disclosure contemplates any suitable bus or interconnect.

Herein, a computer-readable non-transitory storage medium or media may include one or more semiconductor-based or other integrated circuits (ICs) (such, as for example, field-programmable gate arrays (FPGAs) or application-specific ICs (ASICs)), hard disk drives (HDDs), hybrid hard drives (HHDs), optical discs, optical disc drives (ODDs), magneto-optical discs, magneto-optical drives, floppy diskettes, floppy disk drives (FDDs), magnetic tapes, solid-state drives (SSDs), RAM-drives, SECURE DIGITAL cards or drives, any other suitable computer-readable non-transitory storage media, or any suitable combination of two or more of these, where appropriate. A computer-readable non-transitory storage medium may be volatile, non-volatile, or a combination of volatile and non-volatile, where appropriate.

1. A method comprising, by one or more computing systems:
accessing a first image depicting an oral cavity associated with an animal;
detecting, based on one or more machine-learning models, a plurality of teeth associated with the animal from the first image;
identifying, based on the one or more machine-learning models, each of the detected teeth based on a numbering protocol;
determining, for each of the identified teeth based on the one or more machine-learning models, whether the tooth is healthy or has any dental pathology;
localizing each tooth that has any pathology based on the numbering protocol; and
generating a first report comprising a localization of each tooth that has any pathology.

2. The method of claim 1, wherein the first image comprises an X-ray image.

3. The method of claim 1, wherein the first image is based on PNG format or DICOM format.

4. The method of claim 1, further comprising:
determining a quadrant for the first image based on the numbering protocol.

5. The method of claim 1, further comprising:
determining a view for the first image based on whether there is a composition of quadrants or not, wherein the view comprises a lateral view or an occlusal view.

6. The method of claim 1, wherein detecting the plurality of teeth comprises:
determining a plurality of box-coordinates for all possible teeth on the first image; and
calculating a probability score for each of the possible teeth based on the box-coordinates, wherein the probability score indicates a likelihood of the corresponding possible tooth being a tooth.

7. The method of claim 1, further comprising:
segmenting the plurality of detected teeth based on the one or more machine-learning model, wherein the segmentation comprises generating a tooth boundary and a masked tooth without background for each of the plurality of detected teeth.

8. The method of claim 1, wherein the numbering protocol is based on Triadan system.

9. The method of claim 1, wherein identifying each of the detected teeth is based on contextual information associated with each of the detected teeth.

10. The method of claim 1, wherein the one or more machine-learning models comprise a first machine-learning model configured for identifying maxilla teeth and a second machine-learning model configured for identifying mandible teeth.

11. The method of claim 1, further comprising:
determining, for each localized tooth, one or more pathologies associated with the tooth.

12. The method of claim 1, further comprising:
determining, for at least one of the one or more pathologies associated with each tooth, a level of grading.

13. The method of claim 1, further comprising:
determining, based on the one or more machine-learning models, the first image comprises diagnostic information associated with dental pathology detection, wherein the diagnostic information is based on one or more dental structures.

14. The method of claim 1, wherein the one or more dental structures are associated with a particular quadrant.

15. The method of claim 1, wherein the one or more dental structures are associated with a particular dental pathology.

16. The method of claim 1, further comprising:
determining, based on the one or more machine-learning models, that the first image requires an alignment;
determining, based on the one or more machine-learning models, a degree to rotate the first image for the required alignment; and
rotating, based on the one or more machine-learning models, the first image by the determined degree.

17. The method of claim 1, wherein the one or more computing systems are associated with a cloud computing system, and wherein the method further comprises:
receiving, at the cloud computing system, a plurality of second images depicting the oral cavity associated with the animal;
processing the plurality of second images in a parallel manner, wherein processing each of the plurality of second images comprises:
using the one or more machine-learning models in a parallel manner to:
detect a plurality of teeth associated with the animal from each second image;
identify each of the detected teeth based on the numbering protocol;
determine, for each of the identified teeth, whether the tooth is healthy or has any dental pathology; and
localize each tooth that has any pathology based on the numbering protocol; and
generating a second report based on the first report and processing results of the plurality of second images.

18. The method of claim 1, wherein processing the plurality of second images in the parallel manner is based on logic generated based on one or more finite state machines.

19. One or more computer-readable non-transitory storage media embodying software that is operable when executed to:
access a first image depicting an oral cavity associated with an animal;
detect, based on one or more machine-learning models, a plurality of teeth associated with the animal from the first image;
identify, based on the one or more machine-learning models, each of the detected teeth based on a numbering protocol;
determine, for each of the identified teeth based on the one or more machine-learning models, whether the tooth is healthy or has any dental pathology;
localize each tooth that has any pathology based on the numbering protocol; and
generate a first report comprising a localization of each tooth that has any pathology.

20.-36. (canceled)

37. A system comprising: one or more processors; and a non-transitory memory coupled to the processors comprising instructions executable by the processors, the processors operable when executing the instructions to:
access a first image depicting an oral cavity associated with an animal;
detect, based on one or more machine-learning models, a plurality of teeth associated with the animal from the first image;
identify, based on the one or more machine-learning models, each of the detected teeth based on a numbering protocol;
determine, for each of the identified teeth based on the one or more machine-learning models, whether the tooth is healthy or has any dental pathology;
localize each tooth that has any pathology based on the numbering protocol; and
generate a first report comprising a localization of each tooth that has any pathology.

38.-54. (canceled)
